(19) United States
(12) Patent Application Publication
(10) Pub. No.: US 2021/0236737 A1
(43) Pub. Date: Aug. 5, 2021

(54) PRESSURE SENSITIVE NEEDLE POSITIONING DEVICES, RELEASE MECHANISMS, AND METHODS

(71) Applicant: VIRGINIA COMMONWEALTH UNIVERSITY, Richmond, VA (US)

(72) Inventors: Alexandria Ritchie, Chesterfield, VA (US); Bennett Ward, Midlothian, VA (US); Hilton Bennett, Richmond, VA (US); Matthew Leccadito, Yorktown, VA (US); Patrick Jones, Richmond, VA (US); Aayushi Agarwal, Aldie, VA (US)

(21) Appl. No.: 17/050,573

(22) PCT Filed: Apr. 26, 2019
(86) PCT No.: PCT/US2019/029308

§ 371 Date: Oct. 26, 2020

Related U.S. Application Data
(60) Provisional application No. 62/662,815, filed on Apr. 26, 2018.

(51) Int. Cl. A61M 5/315

(52) U.S. Cl. CPC A61M 5/31501; A61M 2205/3344; A61M 2205/583; A61M 2210/1003; A61M 2205/581; A61M 2005/3151; A61M 2205/582

(57) ABSTRACT

A device for facilitating positioning of a needle during a medical procedure may include a needle receiver configured for removable/reversible attachment of a proximal end of a needle; a stop set back from a distal end of the needle receiver and against which a needle received by the needle receiver abuts; and a trigger configured to move the needle receiver relative to the stop when activated by a user such that the stop forces detachment between the needle receiver and the needle. The device may monitor the pressure at the distal end of the needle as the needle is advanced and provide indication to a user when a predetermined pressure or pressure differential is reached.

### CROSS-REFERENCE TO RELATED APPLICATIONS

This application or patent claims the benefit of U.S. Provisional Patent App. No. 62/662,815, filed Apr. 26, 2018, the complete contents of which are herein incorporated by reference.

### FIELD OF THE INVENTION

The invention generally relates to medical procedures involving needles and, in particular, the positioning of needles in applications such as but not limited to anesthesiology, radiology, obstetrics, and emergency medicine.

### BACKGROUND

Hospitals spend approximately two billion dollars a year in post-op patient care for misplaced and failed needle placements, such as when an epidural needle punctures the dura.

A prevalent current method of administering epidurals involves the use of a glass syringe which the user taps on to gauge the air resistance in the syringe. A loss of air resistance signifies that the needle has reached the epidural space. This method is highly subjective and therefore prone to high levels of human error.

U.S. Pat. No. 8,282,565 B2 describes a tool and method of positioning and delivering medical devices in a subject. The pressure within a needle is monitored manometrically or otherwise sensed as the needle is advance towards the subject's pericardial space. By reading the pressure within the needle while it is advanced, the clinician is able to know he or she is avoiding insertion of the needle into organs or spaces not intended to be the target location.

U.S. Pat. No. 9,358,038 B2 describes methods and devices for detecting positioning of a probe in a tissue of a patient. A change in pressure is detected about the distal portion of the coupled probe as it is advanced. The detected pressure change indicates probe positioning in the patient's spinal canal.

Existing solutions have failed to cure standing problem with needle positioning in patients. The above-identified patent literature, for example, recognize a desirability of using pressure to assess the location of a needle tip within a patient as it is being advanced through different tissue structures. However, these prior solutions fail to account for additional sliding or mispositioning of the needle once it has reached the desired tissue or space within the patient. Existing solutions wrongly assume that once the needle reaches a desired location, the risks to the patient and the risk of needle misplacement have passed, when in fact these risks persist and may even be heightened. A need persists for means by which needles can be accurately guided to a target location within tissue or internal space of a patient and then maintained in that location once it is reached.

### SUMMARY

According to an aspect of some exemplary embodiments, devices and methods for positioning needles during medical procedures include means for feedback (e.g., numerical, visual, haptic, etc.) as the needle is advanced to indicate the location of the needle tip as well as means for preventing further sliding or mispositioning of the needle once it reaches the desired position/location within the subject. Some exemplary embodiments make the epidural administration process more effective and lower risk for the patient while preventing the needle from moving further into the patient's spine upon detachment of the insertion assistance device.

According to an aspect of some embodiments, during administration of an epidural, exemplary devices and methods reduce or eliminate the risk of human error which may lead to puncturing the dura.

An exemplary device and its use may be described as follows. A simple on/off switch powers on the portable device. A screen lights up and displays information related to the pressure and/or pressure differential at a nozzle which is configured to receive a needle. The provider (e.g., doctor or nurse) attaches an epidural needle to the nozzle such that the proximal end of the needle butts up against a needle bumper (i.e., stop). The provider then advances the needle into the patient by pushing against the thumb tubs (a form of grip). Some fingers may be used to also stabilize the needle if desired. The screen shows changes in pressure and the related differential as the needle passes through different layers of the body. In effect the provider is given the ability to navigate the needle without the assistance of ultrasound by instead sensing pressure variations or differentials through a body. When the epidural space is reached the screen flashes, thereby alerting the provider the epidural space has been reached. The provider may stop advancing the needle. At this stage the needle insertion device may be removed. The provider can release the needle without dislodging it (advancing it further into the patient or pulling it partially back out of the patient) by squeezing the trigger. The device is thereby freed of the needle and the provider can continue the medical procedure according to customary practice.

Some exemplary embodiments comprise a trigger mechanism which provides separation between a needle and an apparatus used to guide the needle as it is being positioned. The separation is such that it minimizes or eliminates risk of moving the needle accidentally. The separation avoids any need for a user to contact the needle. The separation avoids any need for a user to directly apply force to the needle. Though two handed operation may be more convenient, the separation may even be performed single handedly by a user if desired.

According to a further aspect of some embodiments, the needle employed may be a conventional needle (e.g., epidural needle) already on the market. After the positioning device is removed, the medical practitioner may proceed with known uses of the needle (e.g., injecting an anesthetic into the subject). In alternative embodiments, the positioning device may be used to assist in the delivery or extraction of fluids to/from the patient using the needle.

### BRIEF DESCRIPTION OF THE DRAWINGS

FIG. 1 is an exemplary device for facilitating positioning of a needle during a medical procedure with a needle temporarily affixed thereto.

FIG. 2 is the exemplary device shown in an exemplary handheld position by a user.

FIG. 3 is a perspective view of the exemplary device.

FIG. 4 is a perspective view of the device with half the housing removed, thereby showing internal aspects of the device.

FIG. 5 is a top down cross-sectional view of the device including its release mechanism.

FIG. 6 is an exemplary release mechanism.

FIG. 7 is a rear perspective view showing a display of an exemplary device.

FIGS. 8A and 8B are cross-sectional views showing haptic feedback devices integrated with the device.

FIGS. 9A and 9B are an exemplary alternative housing/body configuration.

FIG. 10 is an exemplary method of positioning a needle in a subject during a medical procedure.

FIG. 11 is an exemplary process executed or executable by exemplary devices.

### DETAILED DESCRIPTION

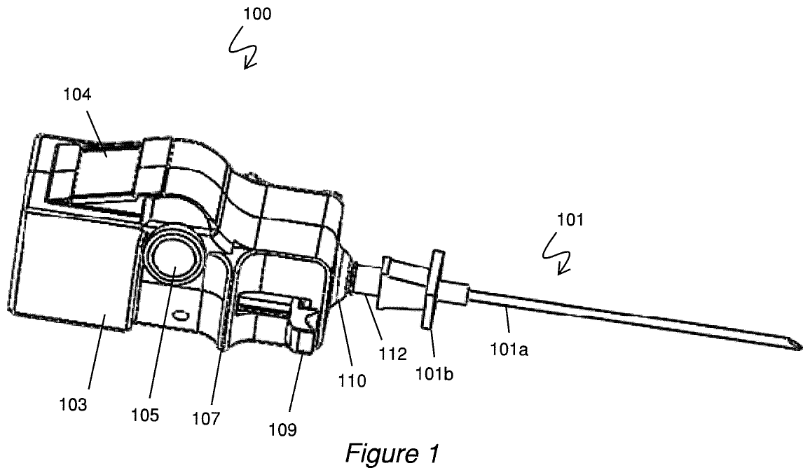

FIG. 1 depicts a device 100 for facilitating positioning of a needle 101 during a medical procedure. The device 100 may comprise, for example, a body 103, user interface 104, switch 105 (e.g., for powering on/off), grip 107, trigger 109, stop 110, and needle receiver 112. The device 100 may be a pressure sensitive needle positioning device. For instance, the device 100 may further include a pressure sensor (e.g., a differential pressure sensor) configured to sense a pressure or pressure differential present at a distal end of the needle receiver 112 or distal end of a needle attached thereto. It will of course be appreciated that “needle” as used herein may be used to refer to a device comprising both tissue penetrating means 101a as well as other components such as external adapter 101b. A needle may for example comprise hollow cannula with proximal tabs for interaction with and support by middle and ring fingers.

In some exemplary embodiments, the grips 107 are configured as tabs that are arranged on opposite sides of the body 103, e.g., on the left side and the right side. As depicted the needle 101 is almost but not completely installed on or attached to the device 100, thereby permitting view of the needle receiver 112. In a fully attached state, the needle 101 may fully abut the stop 110. Physical contact between the proximal end of the needle 101 and the distal end of the stop 110 provides both physical and optical confirmation that the needle 101 is fully secured to the device 100. The needle receiver 112 may be configured (in size, material, and shape, for example) to frictionally engage with the proximal end of the needle 101 to retain the needle in the abutting position in an absence of forces besides gravity. The abutment of the stop 110 with the needle 101 and/or the friction fit between the needle receiver 112 and the needle 101 may form a pressure tight seal (e.g., airtight) between the inner space of the needle and an inner space of the needle receiver 112, discussed in greater detail below.

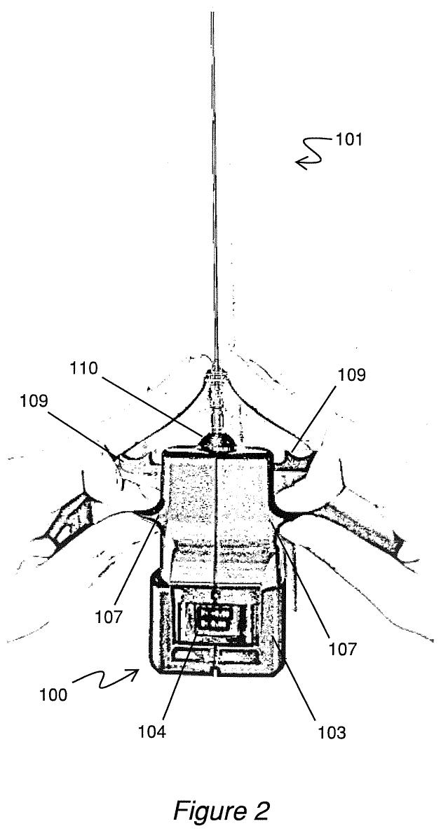

FIG. 2 shows the device 100 as may be held by a user. As a general matter, body 103 is configured to be handheld, especially with respect to size and weight. As depicted in FIG. 2, device 100 could be usable with a single handle, but two handed operation generally gives the greatest amount of control and is therefore preferred in most instances. As depicted in FIG. 2, the grips 107 are each sized and positioned with respect to the remainder of the body 103 to allow the grips to be held between a thumb and pointer finger of the same hand. Thus a left grip 107 is held between the thumb and pointer finger of the left hand, and a right grip 107 is held between the thumb and pointer finger of the right hand. The grips 107 are arranged at a midpart of the body 103 spaced apart from the stop 110 at such distance as to allow the user (e.g., an adult of average hand size) to contact a proximal end of the needle 101 with the middle and/or ring fingers while simultaneously gripping the grips 107 with the thumb and pointer fingers. The middle and ring fingers may be used to stabilize the needle.

Users are generally humans. Exemplary devices and methods are especially well disposed for use by clinicians and medical practitioners such as but not limited to certified registered nurse anesthetists (CRNAs), nurse anesthesiologists, and doctors. This being said, laypersons may also be users of some embodiments. Subjects who administer medicine or therapeutics on a regular basis in a non-medical facility like the home may benefit from a device 100, too.

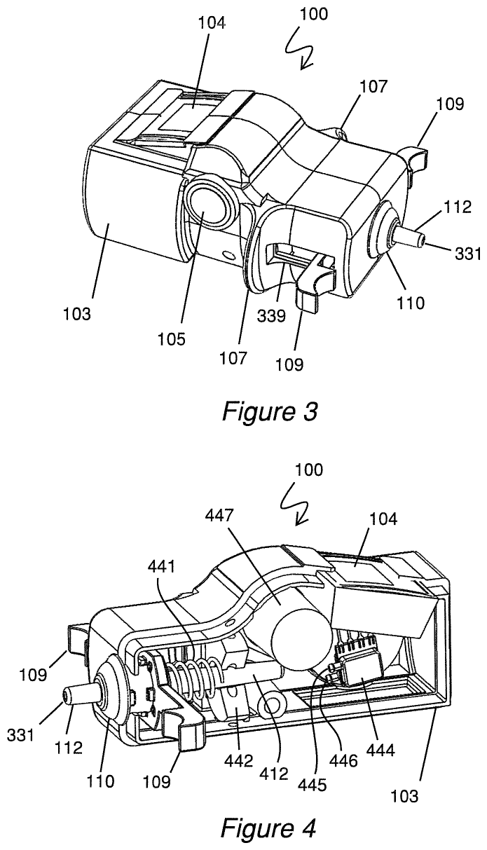

FIG. 3 shows a device 100 without a needle attached to the needle receiver 112. The needle receiver 112 may be the distalmost part of the device 100. This arrangement permits the entirety of any needle which is attached the needle receiver 112 to be arranged in front of the user and in front of the device 100. The distal (e.g., distalmost) end of the needle receiver may be or comprise a nozzle 331. The needle receiver 112, and in particular the nozzle 331, may be configured as a male adapter sized and shaped to mate with a proximal end of a needle which is the female adapter. The nozzle 331 is configured and positioned so as to be in fluid communication with a needle that is received by needle receiver 112. As a result the pressure at the nozzle 331 matches the pressure in an attached needle.

FIG. 3 further shows clearly a track 339 for guiding movement of a trigger 109. A trigger may take a variety of physical configurations provided it remains suitable for “triggering” by a user. An exemplary trigger may be but is not limited to button, or a projection or protrusion such as a tab. A trigger may for example comprise one or two tabs. As used herein the singular “trigger” may refer to either of a singular structure (e.g., a single tab or single button) or multiple structures (e.g., multiple tabs or multiple buttons). In the illustrated device 100, the trigger 109 is a projection or protrusion that is linearly moveable (displaceable) along a distal proximal axis parallel with the axis of a needle received by the needle receiver 112. The track 339 sets limits to the displacement distance of the trigger 109 in both the distal direction and the proximal direction. The trigger 109 is activatable by a squeezing or pinching action with respect to the grips 107. Alternatively, a trigger 109 configured as a button or other structure which may be activated with a pushing action or mere contact action that triggers a linear actuator to complete the needle release with minimal exertion (and minimal risk of needle perturbation) from the user. A non-limiting example of an acceptable linear actuator for some embodiments is the Mini Linear Actuator available from Firgelli Automations having an actuation speed of 1 inch/second.

FIG. 4 is a view of a device 100 in which half of the housing 103 has been removed to permit view of internal components of the device. Within the device 100 is a biasing member 441 such a spring (coiled or otherwise). The biasing member 441 is configured to bias the needle receiver 112 with respect to the stop 110 such that a distal end of the needle receiver 112 is maximally displaced from the stop 110 prior to activation of the trigger 109. The trigger 109 and needle receiver 112 may have a fixed spatial arrangement with respect to one another. Activation of the trigger 109 compresses the spring and draws the distal end of needle receiver 112 toward the stop 110. This retraction has the effect of forcing detachment of a needle from the needle receiver 112, provided of course that a needle was attached at the time of the trigger activation. A spring stop 442 is arranged within the device 100 and serves as a physical body against which the biasing member 441 may be compressed. An exemplary spring compression weight is 25 lb or less. This provides adequate resistance to accidental displacement such as by bumping while permitting user activation solely with forces exerted by hand. A hole or keyway through the spring stop 442 may be provided to allow an extension 412 of the needle receiver 112 to pass through the spring stop 442. The keyway may also support the extension 412 in such a way that it stabilizes the needle receiver 112, helping maintain its position along a single displacement axis with relatively tight tolerances. The spring stop 442 doubles as physical barrier that separates moving components on one side from sensitive electronic and sensing components on the opposite side. The sensing components are described next.

FIG. 4 shows pressure sensor 444 with inlet ports 445 and 446. For clarity of illustration tubing which would ordinarily connect to the inlet port ports 445 and 446 is omitted from illustration. One of the ports 445 is placed in fluid communication with the nozzle 331 via intervening conduits such as tubing (not shown) and extension 412. The other port 446 is placed in fluid communication with ambient pressure or a reference pressure chamber which may be factory set to a predetermined reference pressure. The reference chamber may contain for example air or saline. In some cases the reference chamber may double as a collector of any fluid loss during entry of needle into patient, or during passage through the spinal casing (dura). A separate chamber may also be provided for this purpose. Such chamber may be configured to capture and retain a fluid released by the body during the entry of a needle through various tissues and spaces for disposal, safety or future analysis.

Terminals and wiring are visible in FIG. 4 which allow for sensed pressure information of the pressure sensor 444 to be sent electronically to a circuit board (not shown) where computational functionality may be provided. The circuit board may comprise one or more processors, memory (transitory and/or non-transitory), power regulation, and other circuitry.

FIG. 11 shows a flowchart for an exemplary process executed or executable by exemplary devices like device 100. Upon startup (e.g., after a user activates the power button) the device initializes I/O and the output device (e.g., display) (block 1101). Startup may include establishing a baseline pressure using a reference pressure value such as ambient or some other constant or measurable baseline against which to make a calculation or measurement. The device will then begin to read the pressure sensor (which may interchangeably referred to as a barometric sensor in embodiments where the reference pressure is atmospheric pressure) (block 1102). Depending on the particular sensor for a given embodiment, unit conversion may be employed as necessary. In a prototype embodiment the barometric sensor's output is RAW units, which are converted at block 1103 to millibar and Celsius. Next the device calculates a differential pressure reading (block 1104). Readings at block 1102 are taken continuously, repetitively, and/or cyclically (e.g., upon a predetermined or user defined interval), thereby providing a stream of pressure measures as time advances (and as the needle advances during use in a procedure). Block 1104 may calculate a differential pressure (i.e., a pressure change) reading by taking from a previous pressure reading the latest or most current pressure reading. If the differential pressure reading is zero or substantially zero (e.g., within a set margin of error or tolerance), no output to a user may be necessary to communicate a change since there is no change to report. The device continues to take readings at block 1102 and calculating the differential pressure from the latest readings. If the different pressure calculated at block 1104 is greater than zero, however, a haptic output such as a vibration is output and/or changed from a previous level of haptic output (block 1105). For example, an amount of vibration may increase or it may decrease, or a motionless state may switch to a vibratory state, or a vibratory state may switch to a motionless state.

The haptic response at block 1105 may be used to signify when the needle attached to the device has reached a particular region of tissue. Alternatively or in addition, the haptic feedback device is especially well suited to provide feedback while the needle is advanced yet before the needle reaches a target tissue or region. The haptic response usefully simulates changes in sensation with which clinicians already have some intuitive sense according to convention needle advancing techniques that employ only a glass syringe. A preset or predetermined or user-selected threshold (P_THRESHOLD) is set to correspond with a pressure differential associated with entry to a target tissue or cavity like the epidural space. While the pressure differential from block 1104 remains below the threshold, the haptic response may be updated but the user is not supplied an output signifying the user to halt advancement of the needle. On the other hand, if the differential response meets or exceeds the threshold, the user is alerted such as by a screen alert at block 1106. A flashing screen, for example, is an exemplary screen alert that quickly attracts the user's attention without causing the user to jump or start.

FIG. 4 shows a compartment 447 for containing one or more batteries which may be rechargeable or disposable. The circuit board may contain means or methods of storing and relaying information to a data collection point as a way of collecting patient data and/or as a method of research to validate unknowns regarding the epidural space. The device 100 may comprise capability to transmit data from the device to another location by wifi, lifi, bluetooth, radio frequency, or some other wireless method. The device 100 may be configured to collect and store data for later collection by a separate device or method.

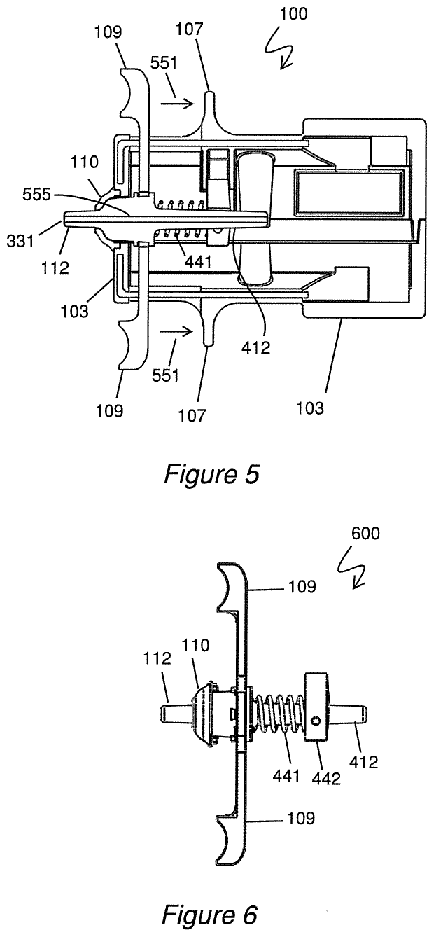

FIG. 5 is another view exposing internal aspects of the device 100. FIG. 5 is a cross-sectional view which bisects the needle receiver 112 to provide a clear view of conduit 555 which runs from the nozzle 331 through extension 412 to fluidically connect with tubing (not shown) and ultimately pressure sensor 444 via port 445 (shown in FIG. 4). In this way a pressure at the nozzle 331 (at the distalmost end of the needle receiver 112) can be sensed by the pressure sensor 444 which may be setback further within the device 100. While this arrangement is exemplary for some embodiments, other configurations may be used in some embodiments. Provided a pressure sensing element of sufficiently small size, the sensor may be arranged at the distal tip of the needle receiver 112, in which case conduit 555 may be omitted or used only for wiring for signal transferal as opposed to a conduit for pressure.

A trigger 109 may be activated by exertion of a force which causes it to move along direction 551. The biasing member exerts a biasing force in opposite the direction 551. FIG. 5 shows clearly the stop 110 as being supported and therefore stationary with respect to the housing 103. The handheld body 103 and the stop 110 are configured to remain motionless when the trigger 109 is activated and thus causes the needle receiver 112 to move relative to the stop 110.

FIG. 6 shows what may collectively be referred to as the release mechanism 600. The release mechanism 600. The release mechanism 600 is configured to detach the pressure sensitive needle positioning device 100 from an attached needle without moving the needle. In some instances release may entail no needle movement whatsoever. In some instances, however, small margins of error or tolerance may be acceptable, depending on the tissue or cavity of the body into which the needle has been inserted. In some instances, margins of e.g. ±0.1 mm may be acceptable margins of error for a no needle movement requirement. Release mechanism 600 comprises needle receiver 112, stop 110, trigger 109, biasing member 441, spring stop 442, and extension 412. Some parts of the release mechanism are configured to move relative to other parts of the release mechanism. As discussed above, the body 103 may rigidly secure the positions of stop 110 and spring stop 442 so they maintain constant positions relative to one another and relative to other components of the release mechanism 600. The release mechanism 600 is activatable without any contact between a user and the needle. The user may instead contact only the trigger 109. The release mechanism 600 may be configured to perform the detachment without a twisting or turning action. That is, neither the needle nor the device need be twisted with respect to the other to effect the release.

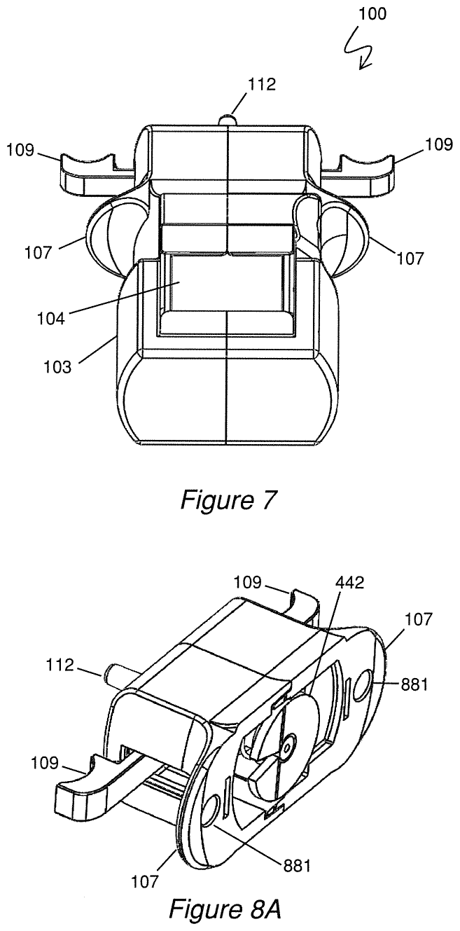

FIG. 7 is a view of device 100 which permits a clear view of user interface 104. In some cases the user interface of a device may be referred to as an output device. An exemplary interface 104 is a display for visual feedback. Additional or alternative to a display, an interface or output device may comprise one or more speakers for audio/auditory feedback, one or more haptic devices (e.g., vibrators, motors, linear actuators) for haptic feedback, and/or other elements. A display may be configured to show a digital numerical readout of the pressure sensed by pressure sensor 444 or a pressure differential, for example. The absolute value of pressure or pressure differential may be provided so a provider is not confused by a negative number. Thus regardless of whether reference pressure is taken from, for example, ambient air, saline, or water, the signage of the output may be the same. A display may be configured to flash when a predetermined pressure or pressure differential is sensed. An exemplary display may include one or more of liquid crystal display (LCD), OLED display, LED display, some combination of these, and/or other display technology. In some embodiments the display may be or include one or more individual LEDs which may be flashed on/off to communicate an alert or other information to a user. A visual notification may be provided when a predetermined pressure is sensed, e.g., the pressure associated with a particular anatomical space such as the spinal canal. Feedback may be continuous or discrete. Haptic feedback may advantageously be configured to mimic or simulate haptic feedback associated with traditional epidural administration techniques.

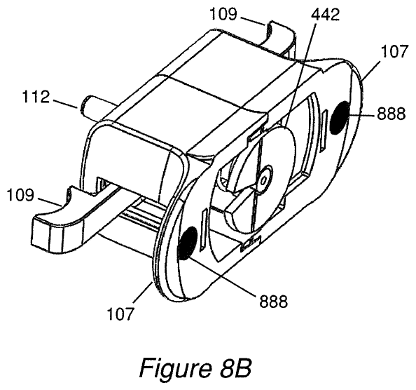

FIGS. 8A and 8B shows a split view of the body taken at grips 107 (here the grips are configured as thumb tabs). The symmetrical holes 881 in the thumb tabs may each house a haptic feedback device 888 such as a vibrating motor/sensor or linear actuator that responds to changes in pressure as the needle is advanced to give the user/provider haptic (e.g., tactile) feedback. A suitable vibration motor (which may sometimes referred to as a micromotor) for some embodiments may be custom built for some embodiments or else commercially acquired. A non-limiting commercially available sample motor has a disk shape with specs of 3V, 1200 rpm, and 10 mm×3 mm dimensions. Such motors are common in phones and pagers, for example. A non-limiting commercially available vibrating sensor is a piezo sensor, such as the LDT1-028L by TE Connectivity. The haptic feedback device 888 may be configured to provide constant or variable haptic feedback based on pressure or density changes. For example, in some configurations (e.g., for some devices or for a setting selected from a plurality of settings of a particular device), a constant vibration setting may be used for all sensed pressures or differentials under a particular threshold (e.g., all pressures under an “epidural space” value). Only when the threshold is reached (e.g., when the needle has entered the epidural space and a corresponding pressure change is sensed) is the vibration magnitude changed (e.g., increased). In an alternative configuration, any change in pressure may be communicated to a user by a corresponding change in vibration intensity (increase or decrease). A unique vibration setting may still be used for a specific pressure condition, like penetration of the needle tip into the epidural space. In short, the pressure gradient experienced as a clinician is holding the device and advancing an (epidural) needle is translated by the device into a tactile response that the clinician can feel in her hand.

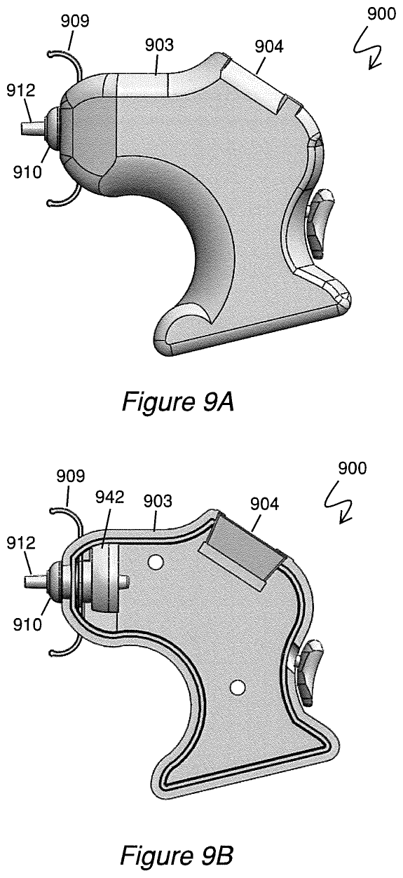

FIGS. 9A and 9B show a device 900 with an alternative housing configuration from that of device 100. Housing 903 is configured with a “pistol” shape. Though the housing differs, device 900 includes many analogous structures to device 100. Device 900 comprises trigger 909, needle receiver 912, (needle) stop 910, and interface 904. Housings such as 103 and 903 may be sterilized, sterilizable, and/or disposable.

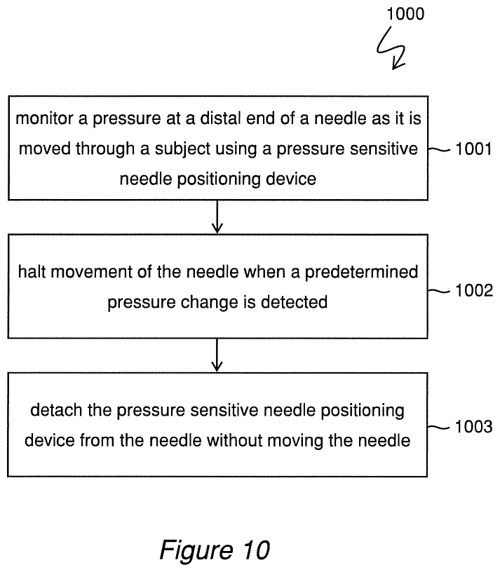

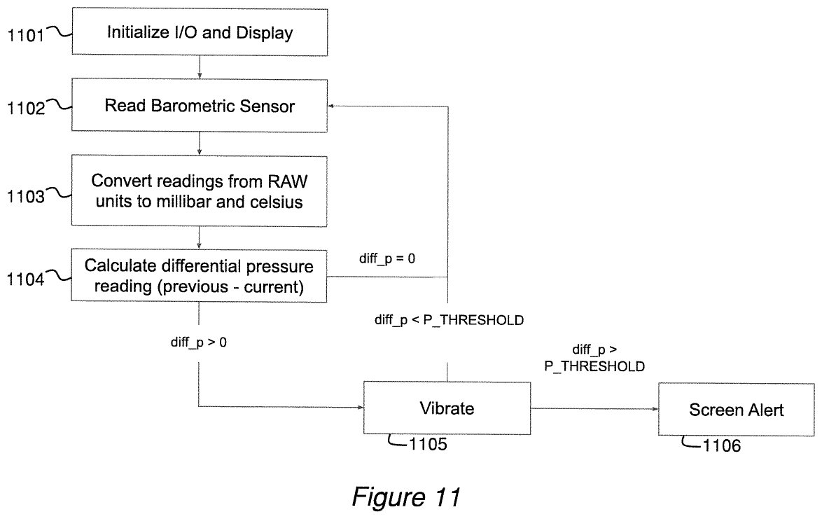

FIG. 10 shows an exemplary method 1000 of positioning a needle in a subject during a medical procedure. Step 1001 comprises monitoring a pressure at a distal end of a needle as it is moved through a subject using a pressure sensitive needle positioning device. The method may involves advancing the needle in e.g. 5 mm increments until the pressure sensor detects the loss of pressure that establishes placement of the needle into the epidural space. Step 1002 comprises halting (e.g., discontinuing, stopping) movement of the needle when a predetermined pressure change is detected. Step 1003 comprises detaching the pressure sensitive needle positioning device from the needle without moving the needle further into the subject (which may run a risk of going past the correct epidural space, for example). Devices 100 and 900 as described above are exemplary pressure sensitive needle positioning devices for performing method 1000. The detaching step 1003 may comprise for example retracting a part of the pressure sensitive needle positioning device into a body of the device. With devices such as device 100 or 900 described above, the detachment step 1003 may be performed without any contact between a user and the needle, thereby eliminating or at least significantly reducing the risk of accidental displacement of the needle into or out of the user during detachment. In some embodiments a very small amount of displacement may still be tolerable, such as 1 mm or less.

A pressure sensing device according to some embodiments quantifies the pressure drop that is experienced when entering the epidural space and alerts the user when the epidural space is reached. Conventional epidural needles often have depth demarcations to assist a medical provider in determining how far the needle has entered a subject's body. Some exemplary devices do not require distance measurements, however, since they may instead rely on a pressure difference between the epidural space and a reference pressure (e.g., atmosphere) to signal penetration of the epidural space. Because conventional needles may be used with exemplary devices of the invention, the conventional depth measurements provided by the needle may continue to be used in addition the pressure sensing techniques of the needle positioning device.

Methods and devices as discussed herein are not necessarily limited to epidurals. Epidural procedures for expectant mothers is one exemplary use case. Another exemplary application is epidurals in any region of the spine for nerve blocks. Nerve blocks are performed for a variety of different procedures including but not limited to shoulder, neck, and lower body procedures. Pain management and lumbar punctures are still further exemplary applications. Still further uses may occur to those of skill in the art in view of the teachings herein.

An exemplary device may comprise a space to catch and store a fluid if released from the spinal column, dura, epidural space or lumbar region, to prevent that fluid from settling into the epidural space or lumbar region.

While exemplary embodiments of the present invention have been disclosed herein, one skilled in the art will recognize that various changes and modifications may be made without departing from the scope of the invention as defined by the following claims.

We claim:

1. A device for facilitating positioning of a needle during a medical procedure, comprising
a pressure sensitive needle positioning device, and
a release mechanism configured to detach the pressure sensitive needle positioning device from an attached needle without moving the needle.

2. The device of claim 1, further comprising an output device configured to give one or more of a visual, audio, and haptic output in response to a pressure or pressure differential being sensed by the pressure sensitive needle positioning device.

3. The device of claim 2, wherein the output device includes a display configured to display pressure or pressure differential information.

4. The device of claim 2, wherein the output device includes a display configured to flash when a predetermined pressure or pressure differential is sensed.

5. The device of claim 1, wherein the output device includes a haptic feedback device configured to provide haptic feedback based on pressure changes.

6. The device of claim 1, wherein the release mechanism is configured to perform detachment without a twisting or turning action.

7. The device of claim 1, wherein the release mechanism is activatable without any contact between a user and the needle.

8. The device of claim 1, wherein the release mechanism comprises
a needle receiver configured for attachment of a proximal end of the needle;
a stop set back from a distal end of the needle receiver and against which a needle received by the needle receiver abuts; and
a trigger configured to move the needle receiver relative to the stop when activated by a user such that the stop forces detachment between the needle receiver and the needle.

9. A device for facilitating positioning of a needle during a medical procedure, comprising
a needle receiver configured for attachment of a proximal end of a needle;
a stop set back from a distal end of the needle receiver and against which a needle received by the needle receiver abuts;
a trigger configured to move the needle receiver relative to the stop when activated by a user such that the stop forces detachment between the needle receiver and the needle;
a pressure sensor configured to sense a pressure or pressure differential present at a distal end of the needle receiver or distal end of a needle attached thereto; and
an output device for providing feedback of the sensed pressure or pressure differential to a user.

10. The device of claim 9, wherein the output device is configured to give one or more of a visual, audio, and haptic output in response to the pressure or pressure differential being sensed by the pressure sensor.

11. The device of claim 10, wherein the output device includes a display configured to display pressure or pressure differential information.

12. The device of claim 10, wherein the output device includes a display configured to flash when a predetermined pressure or pressure differential is sensed.

13. The device of claim 9, wherein the output device includes a haptic feedback device configured to provide haptic feedback based on pressure changes.

14. The device of claim 9, further comprising a handheld body configured to be held by a medical practitioner during insertion of the needle into a subject.

15. The device of claim 14, wherein the handheld body, stop, and needle are configured to remain motionless when the trigger causes the needle receiver to move relative to the stop.

16. The device of claim 9, wherein the abutment of the stop and/or a friction fit between the needle receiver and the needle form an airtight seal.

17. The device of claim 9, further comprising a biasing member configured to bias the needle receiver with respect to the stop such that a distal end of the needle receiver is maximally displaced from the stop prior to activation of the trigger.

18. The device of claim 9, wherein the trigger and needle receiver have a fixed spatial arrangement with respect to one another.

19. The device of claim 9, wherein the trigger comprises one or two tabs.

20. The device of claim 9, wherein the trigger is activatable by a squeezing or pinching action.

21. The device of claim 9, wherein the needle receiver is a nozzle.

22. The device of claim 9, wherein the needle receiver is configured as a male adapter and the proximal end of a needle comprises a female adapter configured to mate with the male adapter.

23. A device for facilitating positioning of a needle during a medical procedure, comprising
a needle receiver configured for attachment of a proximal end of a needle;
a stop set back from a distal end of the needle receiver and against which a needle received by the needle receiver abuts; and
a trigger configured to move the needle receiver relative to the stop when activated by a user such that the stop forces detachment between the needle receiver and the needle.

24. The device of claim 23, further comprising a handheld body configured to be held by a medical practitioner during insertion of the needle into a subject.

25. The device of claim 24, wherein the handheld body, stop, and needle are configured to remain motionless when the trigger causes the needle receiver to move relative to the stop.

26. The device of claim 23, wherein the abutment of the stop and/or a friction fit between the needle receiver and the needle form an airtight seal.

27. The device of claim 23, further comprising a biasing member configured to bias the needle receiver with respect to the stop such that a distal end of the needle receiver is maximally displaced from the stop prior to activation of the trigger.

28. The device of claim 23, wherein the trigger and needle receiver have a fixed spatial arrangement with respect to one another.

29. The device of claim 23, wherein the trigger comprises one or two tabs.

30. The device of claim 23, wherein the trigger is activatable by a squeezing or pinching action.

31. The device of claim 23, wherein the needle receiver is a nozzle.

32. The device of claim 23, wherein the needle receiver is configured as a male adapter and the proximal end of a needle comprises a female adapter configured to mate with the male adapter.

33. A device for facilitating positioning of a needle during a medical procedure, comprising
a pressure sensitive needle positioning device, and
a haptic feedback device configured to provide a constant or variable tactile feedback response based on pressure changes.

34. The device of claim 33, further comprising a chamber configured to capture and retain a fluid released by the body during the entry of a needle through various tissues and spaces for disposal, safety or future analysis.

35. A method of positioning a needle in a subject during a medical procedure, comprising
monitoring a pressure or pressure change at a distal end of a needle as it is moved through a subject using a pressure sensitive needle positioning device;
halting movement of the needle when a predetermined pressure or pressure change is detected; and
detaching the pressure sensitive needle positioning device from the needle without moving the needle further into the subject.

36. The method of claim 35, wherein the step of detaching is performed without any contact between a user and the needle.

37. The method of claim 35, wherein the step of detaching comprises retracting a part of the pressure sensitive needle positioning device into a body of the device.

38. The method of claim 35, wherein the step of detaching is performed without moving the needle.
